(19) United States
(12) Patent Application Publication
(10) Pub. No.: US 2010/0035949 A1
(43) Pub. Date: Feb. 11, 2010

(54) MICROEMULSION DOSAGE FORMS OF VALSARTAN AND METHODS OF MAKING THE SAME

(76) Inventors: Yatindra Joshi, Princeton, NJ (US); John J. Kennedy, Hillsborough, NJ (US); Ping Li, Basking Ridge, NJ (US); Alan E. Royce, Saylorsburg, PA (US); Robert F. Wagner, Hillsborough, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/517,105

(22) PCT Filed: Dec. 3, 2007
(86) PCT No.: PCT/US07/86226

§ 371 Date: Jun. 1, 2009

Related U.S. Application Data
(60) Provisional application No. 60/868,604, filed on Dec. 5, 2006.

(51) Int. Cl. A61K 31/41; A61P 9/12; A61P 9/00

(52) U.S. Cl. 514/381

(57) ABSTRACT

A drug delivery system, e.g., microemulsion preconcentrate, that spontaneously forms a microemulsion when brought in contact with an aqueous medium. The drug delivery system contains valsartan, a hydrophilic component, a lipophilic component and a surfactant. A particularly useful hydrophilic component in the system is a polymer that is solid at room temperature, e.g., solid PEG.

### BACKGROUND OF THE INVENTION

### Field of the Invention

The present invention relates to a drug delivery system that includes a microemulsion preconcentrate of valsartan, in a liquid, solid or semisolid carrier. The system forms an emulsion, e.g., a microemulsion when brought in contact with an aqueous medium, e.g., water or the gastric juices of the gastrointestinal tract.

### BACKGROUND OF THE INVENTION

The development of microemulsion dosage formulations of certain active ingredients is challenging. When formulating microemulsion dosage formulations, the objective is to provide an increased release of valsartan and increased oral bioavailability of valsartan in a patient as compared to known solid oral dosage forms of valsartan. Development of microemulsion dosage formulations that have improved bioavailability to the known oral dosage forms of valsartan is challenging due to the multiplicity of challenges arising from pharmacokinetic aspects of oral drug delivery.

For example, valsartan has an oral bioavailability of only about 25% with a wide range of 25-40% in humans with large inter- and intra-subject variabilities. Valsartan also has pH dependent solubility whereby it ranges from very slightly soluble in an acidic environment to soluble in a neutral environment of the gastrointestinal tract. The permeability of valsartan is low and also pH dependent where it decreases as environmental pH increases from acidic to a neutral pH values in the gastrointestinal track. As a result of these complex biopharmaceutical properties, development of a more releasable and bioavailabe dosage form of valsartan with less inter- and intra-subject variability is challenging.

Accordingly, a microemulsion dosage formulation of valsartan which has enhanced release and bioavailability properties with less inter- and intra-subject variability would be desirable.

### SUMMARY OF THE INVENTION

A drug delivery system, or pharmaceutical composition, is disclosed herein. The drug delivery system contains valsartan, within a carrier being in the form of a preconcentrate, i.e., microemulsion preconcentrate, that contains a lipophilic component, hydrophilic component, surfactant, and optionally additional excipients.

The microemulsion preconcentrate, at room temperature is liquid, solid or semisolid. When the drug delivery system is brought into contact with an aqueous medium, e.g., gastric juices, it forms a microemulsion with the aqueous medium. For example, an o/w microemulsion is formed with the aqueous medium being the external phase. The internal phase contains at least the lipophilic component, and the drug being delivered may be present in or mixed with the internal phase or at the surface of the internal phase. Preferably, valsartan is in the free acid form.

In one exemplary embodiment of the present invention, the lipophilic component is a liquid lipophilic component, e.g., an oil. In a particular aspect of the present invention, the hydrophilic component is a polymer that is solid at room temperature.

Particularly useful as a solid polymer are solid polyoxyethylene glycols. Examples of solid polyethylene glycols (PEGs) include, but are not limited to, PEG 1450, PEG 3350, PEG 4000, PEG 8000 and combinations and mixtures thereof. In another aspect of the present invention, the emulsion formed is a microemulsion having particles that have a mean particle size of about 50 nm to about 300 nm.

In a second preferred aspect, the present invention is directed to a process for preparing a microemulsion preconcentrate containing valsartan. Such a process, e.g., includes the steps of bringing valsartan and a liquefied carrier having a surfactant, a lipophilic component and a hydrophilic component into intimate admixture to form a pharmaceutical composition. The resulting composition is, e.g., solid or semisolid at room temperature. Preferably, the mixture is placed within a capsule for oral delivery. The capsule is preferably enteric coated. The pharmaceutical composition is then subsequently brought into contact with an aqueous medium to form a microemulsion.

In certain preferred embodiments of this invention, the amount of valsartan employed in such pharmaceutical compositions preferably ranges from about 20 mg to about 640 mg, and more preferably is 80 mg or 160 mg.

Yet another preferred aspect of the invention is directed to a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering a pharmaceutical composition of the present invention to a subject in need of such treatment.

In another aspect of the present invention there is provided a use of a pharmaceutical composition of the present invention for the manufacture of a medicament for the treatment of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure.

A pharmaceutical composition for the treatment of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising valsartan, a lipophilic component, hydrophilic component and a surfactant.

### DETAILED DESCRIPTION

The present invention relates to a drug delivery system whereby a microemulsion preconcentrate can be delivered in a liquid, solid or semisolid form. The drug delivery system includes valsartan in a carrier that comprises a lipophilic component, a surfactant and a hydrophilic component. When the drug delivery system is brought into contact with an aqueous medium, an emulsion, especially a microemulsion, spontaneously forms. In particular, a microemulsion forms in the digestive tract of a mammal when the delivery system of the present invention is orally ingested. In addition to the aforementioned components, the drug delivery system can also optionally contain other excipients, such as buffers, pH adjusters, stabilizers, cosurfactants, fillers, acidifiers and other adjuvants recognized by one of ordinary skill in the art to be appropriate for such a pharmaceutical use.

As used herein, the term “drug delivery system” means a pharmaceutical composition containing a drug to be administered to a mammal, e.g., a human. A pharmaceutical composition is “pharmaceutically acceptable” which refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

Valsartan ((S)-N-valeryl-N-{[2′-(1H-tetrazole-5-yl)-biphenyl4-yl]-methyl}-valine) suitable for use in the present invention can be purchased from commercial sources or can be prepared according to known methods. For example, the preparation of valsartan is described in U.S. Pat. No. 5,399,578, the entire disclosure of which is incorporated by reference herein. Valsartan may be used for purposes of this invention in its free form.

Valsartan is employed in an amount typically ranging from about 20 mg to about 640 mg, preferably from about 40 mg to about 320 mg, more preferably from about 80 mg to about 320 mg, and most preferably is about 80 mg or about 160 mg. The amount of valsartan noted above refers to the amount of free valsartan present in a given microemulsion dosage form.

As used herein, the term “carrier” refers to the pharmaceutically acceptable composition that transports the drug across the biological membrane or within a biological fluid. The carrier, of the present invention, comprises a lipophilic component, a hydrophilic component and a surfactant. The carrier of the present invention is capable of spontaneously producing a microemulsion or colloidal structures, when brought in contact, dispersed, or diluted, with an aqueous medium, e.g., water, fluids containing water, in vivo media in mammals, such as the gastric juices of the gastrointestinal tract. The colloidal structures can be solid or liquid particles including droplets, micelles and nanoparticles. The carrier, e.g., is a microemulsion preconcentrate (as described in more detail below).

As used herein, the term “microemulsion” refers to a clear or slightly cloudy colloidal dispersion that is formed spontaneously or substantially spontaneously when its components are brought into contact with an aqueous medium. A microemulsion is thermodynamically stable and contains dispersed particles of a mean diameter less than about 300 nm, e.g., less than about 250 nm, less than 150 nm, less than 100 nm, greater than about 2-4 nm as measured by standard light scattering techniques, e.g., using a MALVERN ZETASIZER 3000 particle characterizing machine. Microemulsions, e.g., are thermodynamically stable, e.g., for at least 15 minutes, or up to four hours or even 24 hours or longer.

Microemulsions can offer greater ease of preparation due to spontaneous formation, thermodynamic stability and elegant aesthetics. Microemulsions improve the delivery of the drug because they can increase drug loading, enhance penetration, reduce particle size, improve particle size uniformity, increase dissolution rate, increase bioavailability and reduce inter- and intra-individual variability in drug pharmacokinetics as compared to traditional coarse emulsions. As used herein, the term “bioavailable”, with reference to a composition, means that composition provides a maximum concentration of the drug in that composition in a use environment that is at least 1.5-fold that of a control comprising an equivalent quantity of the undispersed drug.

As used herein, the term “microemulsion preconcentrate” means a composition, or preconcentrate, which spontaneously forms a microemulsion, e.g., an o/w microemulsion, in an aqueous medium, in water, e.g., on dilution of 1:1 to 1:300, or from 1:1 to 1:70, or from 1:1 to 1:10 or in the gastrointestinal fluids after oral application.

The relative proportions, within the microemulsion preconcentrate, of the lipophilic component, the hydrophilic component and the surfactant lie within the “microemulsion” region on a standard three-way plot graph. Such graphs, or phase diagrams, can be generated in a conventional manner by one of ordinary skill in the art. For example, as described in Great Britain Patent No. 2,222,770, which is hereby incorporated by reference in its entirety.

A microemulsion preconcentrate, comprises a lipophilic component, a hydrophilic component and a surfactant. The hydrophilic component and the surfactant together in the drug delivery system can comprise up to 95% by weight of the composition of the carrier, e.g., 80%.

As used herein, the term “solidify” means to make solid or semisolid. “Semisolid” means having the qualities and/or attributes both of the solid and liquid states of matter.

As used herein, the term “lipophilic component” refers to a substance, material or ingredient that is more compatible with oil than with water. A material with lipophilic properties is insoluble or almost insoluble in water but is easily soluble in oil or other nonpolar solvents. The term “lipophilic component” can comprise one or more lipophilic substances. Multiple lipophilic components constitute the lipophilic phase of the microemulsion preconcentrate and form the oil aspect, e.g., in an o/w microemulsion. At room temperature (approximately 25-27° C.), the lipophilic component and lipophilic phase of the microemulsion preconcentrate can be solid, semisolid or liquid. For example, a solid lipophilic component can exist as a paste, granular form, powder or flake.

Examples of solid lipophilic components, i.e., solid or semisolid at room temperature, include, but are not limited to, the following:
1. mixtures of mono-, di- and triglycerides, such as hydrogenated coco-glycerides [melting point (m.p.) of about 33.5° C. to about 37° C.], commercially-available as WITEPSOL H15 from Sasol Germany (Witten, Germany);
2. esters, such as propylene glycol (PG) stearate, commercially-available as MONOSTEOL (m.p. of about 33° C. to about 36° C.) from Gattefossé Corp. (Paramus, N.J.); PEG-2 stearate, commercially-available as HYDRINE (m.p. of about 44.5° C. to about 48.5° C.) from Gattefossé Corp.; cetyl palmitate (m.p. of about 50° C.), commercially-available as CUTINA CP from Cognis Corp. (Hoboken, N.J.);
3. glyceryl fatty acid esters, such as hydrogenated palm/palm kernel oil PEG-6 esters (m.p. of about 30.5° to about 38° C.), commercially-available as LABRAFIL M2130 CS from Gattefossé Corp.;
4. fatty alcohols, such as myristyl alcohol (m.p. of about 39° C.), commercially-available as LANETTE 14 from Cognis Corp.; and
5. polyglycosylated saturated glycerides, such as lauroyl macrogol-32 glycerides (m.p. of about 42-46° C.), commercially available as CELUCIRE 44/14 from Gattefossé Corp. Although GELUCIRE 44/14 is dispersible in water, for the present invention, GELUCIRE 44/14 is a solid lipophilic compound.

Examples of liquid lipophilic components, i.e., liquid at room temperature include, but are not limited to, the following:
1. mixtures of mono-, di- and triglycerides, such as medium chain mono- and diglycerides glyceryl caprylate/caparate, commercially-available as CAPMUL MCM from Abitec Corp. (Columbus, Ohio);
2. esters, such as PG monocaprylate, commercially-available as CAPMUL PG-8 from Abitec Corp.;
3. oils, such as safflower oil, sesame oil, corn oil, castor oil, coconut oil, cotton seed oil, soybean oil, olive oil and mineral oil;
4. essential oils, or any of a class of volatile oils that give plants their characteristic odors, such as spearmint oil, clove oil, lemon oil and peppermint oil;
5. fractions or constituents of essential oils, such as menthol, carvacrol and thymol; and
6. synthetic oils, such as triacetin, tributryin, ethyl butyrate, ethyl caprylateoleic acid, ethyl oleate, isopropyl myristate and ethyl caprylate.

The lipophilic component comprises from about 5% to about 85% by weight of the composition of the carrier, e.g., from about 10% to about 85%, e.g., from about 15% to about 60%, e.g., from about 20% to about 40%.

As used herein, the “hydrophilic component” comprises a hydrophilic component and/or water. A solid hydrophilic component is added in the microemulsion preconcentrate in order to render or help render the microemulsion preconcentrate a solid or semisolid at room temperature. An example of a hydrophilic component is PEG which is the polymer of ethylene oxide that conforms generally to the formula H(OCH₂CH₂)ₙOH in which n represents the average molecular weight of the polymer.

The types of PEG useful in the present invention can be categorized by its state of matter, i.e., whether the substance exists in a solid or liquid form at room temperature and pressure. As used herein, “solid PEG” refers to PEG having a molecular weight such that the substance is in a solid state at room temperature and pressure. For example, PEG having a molecular weight ranging between 1,000 and 10,000 is a solid PEG. Particularly useful solid PEGs are those having a molecular weight between 1,450 and 8,000. Especially useful as a solid PEG are PEG 1450, PEG 3350, PEG 4000, PEG 8000, derivatives thereof and mixtures thereof. PEGs of various molecular weights are commercially-available as the CARBOWAX SENTRY series from Dow Chemicals (Danbury, Conn.). Moreover, solid PEGs have a crystalline structure, or polymeric matrix, which is a particularly useful attribute in the present invention.

In one exemplary embodiment of the present invention, up to eighty percent of the carrier, when liquefied, for example comprising the lipophilic component, surfactant and valsartan, can be incorporated into the hydrophilic component without disturbing the crystalline structure of the hydrophilic component.

Also useful as the hydrophilic component, are PEG derivatives which include, but are not limited to, block co-polymers, such as different poloxamers commercially-available from BASF Corp. (Mt. Olive, N.J.) and Vitamin E TPGS.

Yet another example of a useful hydrophilic component is polyethylene oxide (“PEO”) which is a nonionic homopolymer of ethylene oxide, represented by the formula (CH₂Ch₂O)ₙ, in which n represents the average number of oxyethylene groups. The various grades of PEO are commercially available as POLYOX from Dow Chemicals. At room temperature and pressure, PEO exists in a solid state. PEO, for example, has a molecular weight ranging from about 100,000 to 7,000,000. The hydrophilic component in the present invention can comprise PEG, PEO, and any combinations of the foregoing.

The hydrophilic component may comprise from about 15% to about 90% by weight of the carrier, e.g., from about 20% to about 70%, e.g., from about 30% to about 50%.

In an alternative exemplary embodiment, the hydrophilic component of the carrier consists of a single hydrophilic component, e.g., a solid PEG, e.g., PEG 1450, PEG 3350, PEG 4000 and PEG 8000. In this exemplary embodiment, the hydrophilic phase of the microemulsion component consists of a single hydrophilic substance. For example, if the carrier comprised PEG 3350, the carrier would contain no other hydrophilic substances, e.g., lower alkanols (lower alkyl being C₁-C₄), such as ethanol; or water. Substances that have affinity for both the lipophilic phase and the hydrophilic phase, such as surfactants would not be considered a hydrophilic substance for this exemplary embodiment. Thus, the carrier could contain a surfactant in addition to the single hydrophilic component.

In yet another alternative exemplary embodiment, the hydrophilic component of the carrier consists of a mixture of solid PEGs. For example, the hydrophilic component comprises PEG 1450, PEG 3350, PEG 4000, PEG 8000, derivatives thereof and any combinations and mixtures thereof.

The carrier also comprises one or more surfactants, i.e., a mixture of surfactants; or surface active agents, which reduce interfacial tension. The surfactant can be added to either the hydrophilic or lipophilic phase of the carrier. The surfactant is, e.g., nonionic, ionic or amphoteric. Surfactants can be complex mixtures containing side products or unreacted starting products involved in the preparation thereof, e.g., surfactants made by polyoxyethylation may contain another side product, e.g., PEG. The surfactant or surfactants can have any HLB that is useful in the pharmaceutical arts. For example, the surfactant has a hydrophilic-lipophilic balance (HLB) having a mean HLB value of 8-17, e.g., 10-17. Examples of surfactants types include, but are not limited to, fatty acids; alkyl sulfonates; polyoxyethylene fatty acids; sorbitan derivatives; polyoxyethylene sorbitan fatty acid esters; lecithin; phospholipids; mono-, di- and triglycerides; and mixtures thereof.

Examples of such surfactants include, but are not limited to,
1. reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. Various such surfactants are commercially-available, e.g., the CREMOPHOR series from BASF Corp. (Mt. Olive, N.J.), such as CREMOPHOR RH 40 which is PEG-40 hydrogenated castor oil which has a saponification value of about 50- to 60, an acid value less than about one, a water content, i.e., Fischer, less than about 2%, an n_{D}⁶⁰ of about 1.453-1.457, and an HLB of about 14-16;
2. polyoxyethylene fatty acid esters that include polyoxyethylene stearic acid esters, such as the MYRJ series from Uniqema (New Castle, Del.), e.g., MYRJ 53 having a m.p. of about 47° C. Particular compounds in the MYRJ series are, e.g., MYRJ 53 having a m.p. of about 47° C. and PEG-40-stearate available as MYRJ 52;
3. sorbitan derivatives that include the TWEEN series from Uniqema (New Castle, Del.), e.g., TWEEN 20, TWEEN 40, TWEEN 60 and TWEEN 80;
4. polyoxyethylene-polyoxypropylene co-polymers and block co-polymers or poloxamers, e.g., SYNPERONIC PE/F 87/108/127L44 from Uniqema;
5. polyoxyethylene alkyl ethers, e.g., such as polyoxyethylene glycol ethers of C₁₂-C₁₈ alcohols, e.g., polyoxyl 2-, 10- or 20-cetyl ether or polyoxyl 23-lauryl ether, or polyoxyl 20-oleyl ether, or polyoxyl 2-, 10-, 20- or 100-stearyl ether, as known and commercially-available as the BRIJ series from Uniqema. Particularly useful products from the BRIJ series are BRIJ 58; BRIJ 76; BRIJ 78; BRIJ 35, i.e., polyoxyl 23 lauryl ether; BRIJ 96; and BRIJ 98, i.e., polyoxyl 20 oleyl ether. These products have a m.p. between about 32° C. to about 43° C.;
6. water-soluble tocopheryl PEG succinic acid esters available from Eastman Chemical Co. (Kingsport, Tenn.) with a m.p. of about 36° C.;
7. PEG sterol ethers having, e.g., from 5-35 [CH₂—CH₂—O] units, e.g., 20-30 units, e.g., SOLULAN C24 (Choleth-24 and Cetheth-24) from Chemron (Paso Robles, Calif.);
8. polyglycerol fatty acid esters, e.g., having a range of glycerol units from 4-10, or 4, 6 or 10 glycerol units. For example, particularly suitable are deca-/hexa-/tetra-glyceryl monostearate, e.g., DECAGLYN, HEXAGLYN and TETRAGLYN from Nikko Chemicals (Tokyo, Japan); and
9. alkylene polyol ether or ester, e.g., lauroyl macrogol-32 glycerides and/or stearoyl macrogol-32 glycerides which are GELUCIRE 44/14 and GELUCIRE 50/13 respectively.

The surfactant or mixture of surfactants may comprise from about 1-90% by weight of the carrier, e.g., from 5-85%, e.g., from 10-80%, e.g., from 20-60%, e.g., from 35-55%.

In certain exemplary embodiments of the present invention, the pharmaceutical composition may comprise additional excipients commonly found in pharmaceutical compositions, examples of such excipients include, but are not limited to, cosurfactants, antioxidants, antimicrobial agents, fillers, acidifiers, enzyme inhibitors, stabilizers, preservatives, flavors, sweeteners and other components as described in Handbook of Pharmaceutical Excipients, Rowe et al., Eds., 4^{th} Edition, Pharmaceutical Press (2003),. which is hereby incorporated by reference.

A “cosurfactant”, as used herein, is a surface-active agent that acts in addition to the surfactant by further lowering the interfacial energy but that cannot form micellar aggregates by itself. Cosurfactants can be, for example, hydrophilic or lipophilic. Examples of a cosurfactant include, but are not limited to, cetyl alcohol and stearyl alcohol.

Examples of antioxidants include, but are not limited to, ascorbic acid and its derivatives, tocopherol and its derivatives, butyl hydroxyl anisole and butyl hydroxyl toluene. Vitamin E as α-tocopherol is particularly useful.

Examples of fillers include, but are not limited to, microcrystalline cellulose, silicone dioxide, starch and its derivatives, lactose, dicalcium phosphate and mannitol.

Examples of acidifiers include, but are not limited to, citric acid, succinic acid, fumaric acid, Ascorbic acid, phosphric acid, capric acid, oleic acid, glutamic acid and hydroxypropyl methyl cellulose acetate succinate, cellulose acetate phthalate, cellulose acetate trimellitate, hydroxypropyl methyl cellulose phthalate, carboxymethyl ethyl cellulose and carbomer.

These additional excipients may comprise from about 0.05-50% by weight of the total pharmaceutical composition. Antioxidants, anti-microbial agents, enzyme inhibitors, stabilizers or preservatives typically provide up to about 0.05-1% by weight of the total pharmaceutical composition. Sweetening or flavoring agents typically provide up to about 2.5% or 5% by weight of the total pharmaceutical composition.

The microemulsion preconcentrate can be prepared separately before bringing into intimate admixture with the drug. Alternatively, two or more of the components of the carrier can be mixed together with valsartan.

The spontaneously dispersible microemulsion preconcentrate preferably spontaneously or substantially spontaneously forms an o/w emulsion, e.g., microemulsion, when diluted with an aqueous medium, such as water, to a dilution of 1:1 to 1:300, e.g., 1:1 to 1:70, especially 1:10 to 1:70, more especially, e.g., 1:10, or in the gastrointestinal fluids of a patient after oral administration.

In yet a further aspect, the invention provides a process for the preparing a microemulsion containing valsartan, which process comprises the following steps:
(a) bringing valsartan and a microemulsion preconcentrate comprising a lipophilic component, a surfactant and a hydrophilic component into intimate admixture to form a spontaneously dispersible pharmaceutical composition; and
(b) diluting the spontaneously dispersible pharmaceutical composition in an aqueous medium to form a microemulsion.

The relative proportion of the drug(s), the lipophilic component(s), the surfactant(s) and the hydrophilic component(s) (when present) should lie within the “microemulsion” region on a standard three-way plot graph. The compositions will therefore be of high stability that are capable, on addition to an aqueous medium, of providing microemulsions, e.g., having a mean particle size less than 300 nm, especially less than 200 nm. The microemulsion formed may be administered enterally, e.g., orally, e.g., in the form of a drinkable solution. When the composition of the invention is a microemulsion preconcentrate a unit dosage of the microemulsion preconcentrate can be used to fill orally administrable capsule shells. The capsule shells may be soft or hard capsule shells, e.g., made of gelatine or hydroxylpropylmethyl cellulose. When the capsule shells contact or immersed into an aqueous medium the shells dissolve or disintegrate release the their contents into the aqueous medium allowing the microemulsion to form.

Each unit dosage will suitably contain from 0.1 mg and 1000 mg drug, e.g., 0.1 mg, 1 mg, 5 mg, 10 mg, 15 mg, 20 mg, 25 mg, 40 mg, 50 mg, 80 mg, 100 mg, 160 mg, 200 mg, 250 mg, 300 mg, 320 mg, 400 mg or 500 mg, 640 mg e.g., between 5 mg and 640 mg of drug, e.g., between 10 mg and 100 mg of drug, e.g., between 20 mg and 640 mg of drug. Such unit dosage forms are suitable for administration 1-5 times daily depending upon the particular purpose of therapy, the phase of therapy and the like.

The compositions of the invention exhibit especially advantageous properties when administered orally, e.g., in terms of consistency and high level of bioavailability obtained in standard bioavailability trials.

Pharmacokinetic parameters, e.g., drug substance absorption and measured, e.g., as blood levels, also become surprisingly more predictable and problems in administration with erratic absorption may be eliminated or reduced. Additionally, the pharmaceutical compositions are effective with biosurfactants or tenside materials, e.g., bile salts, being present in the gastrointestinal tract. That is, the pharmaceutical compositions of the present invention are fully dispersible in aqueous systems comprising such natural tensides and thus capable of providing emulsion or microemulsion systems and/or particulate systems in situ which are stable. The function of the pharmaceutical compositions upon oral administration remain substantially independent of and/or unimpaired by the relative presence or absence of bile salts at any particular time or for any given individual. The compositions of this invention may also reduce variability in inter- and intra-patient dose response.

Yet another embodiment of the invention is directed to a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache or chronic heart failure comprising administering a therapeutically effective amount of the pharmaceutical compositions of the present invention to a subject in need of such treatment. In a preferred embodiment, the pharmaceutical compositions are orally administered to the subject.

In another aspect of the present invention there is provided a use of a pharmaceutical composition of the present invention for the manufacture of a medicament for the treatment of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache or chronic heart failure.

Another aspect of the present invention provides a pharmaceutical composition for the treatment of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache or chronic heart failure comprising valsartan, a lipophilic component, hydrophilic component and a surfactant.

Specific embodiments of the invention will now be demonstrated by reference to the following examples. It should be understood that these examples are disclosed solely by way of illustrating the invention and should not be taken in any way to limit the scope of the present invention.

In all of the following examples, valsartan drug substance is added to a suitable container, the oils, surfactants and other excipients are added and the mixture is heated to 60-80° C. and stirred until the drug substance dissolves. Subsequently the mixture may be added into capsules.

### Solid Microemulsion Dosage Form

### EXAMPLE 1

| Ingredient | mg/unit |
|---|---|
| Valsartan | 80 |
| Lauroglycol FCC | 160 |
| Lauroglycol 90 | 50 |
| Tween 20 | 100 |
| Cremophor RH40 | 100 |
| PEG3350 | 150 |

### EXAMPLE 2

| Ingredient | mg/unit |
|---|---|
| Valsartan | 80 |
| Lauroglycol FCC | 150 |
| EtOH | 25 |
| PG | 25 |
| Tween 20 | 100 |
| Cremophor RH40 | 100 |
| PEG3350 | 145 |

### EXAMPLE 3

| Ingredient | mg/unit |
|---|---|
| Valsartan | 80 |
| Spearmint oil | 150 |
| EtOH | 25 |
| PEG400 | 25 |
| Cremophor RH40 | 200 |
| PEG3350 | 150 |
| SiO2 | 20 |

### EXAMPLE 4

| Ingredient | mg/unit |
|---|---|
| Valsartan | 80 |
| VitE TPGS | 150 |
| EtOH | 25 |
| PEG400 | 25 |
| Cremophor RH40 | 200 |
| PEG3350 | 150 |

### EXAMPLE 5

| Ingredient | mg/unit |
|---|---|
| Valsartan | 80 |
| Maisine 35-1 | 150 |
| EtOH | 25 |
| PEG400 | 25 |
| Cremophor RH40 | 200 |
| PEG3350 | 150 |

### EXAMPLE 6

| Ingredient | mg/unit |
|---|---|
| Valsartan | 80 |
| Maisine 35-1 | 150 |
| EtOH | 25 |
| PEG400 | 25 |
| Cremophor RH40 | 200 |
| PEG3350 | 150 |

### EXAMPLE 7

| Ingredient | mg/unit |
|---|---|
| Valsartan | 80 |
| Labfill M2125 | 150 |
| EtOH | 80 |
| Cremophor RH40 | 200 |
| PEG3350 | 150 |
| SiO2 | 20 |

### EXAMPLE 8

| Ingredient | mg/unit |
|---|---|
| Valsartan | 80 |
| Capmul PG8 | 150 |
| EtOH | 80 |
| Cremophor RH40 | 200 |
| PEG3350 | 150 |

### EXAMPLE 9

| Ingredient | mg/unit |
|---|---|
| Valsartan | 80 |
| Capmul MCM | 150 |
| EtOH | 80 |
| Cremophor RH40 | 200 |
| PEG3350 | 150 |

### EXAMPLE 10

| Ingredient | mg/unit |
|---|---|
| Valsartan | 80 |
| Clove oil | 150 |
| EtOH | 25 |
| PEG400 | 25 |
| Cremophor RH40 | 200 |
| PEG3350 | 150 |
| SiO2 | 20 |

### Liquid Microemulsion Dosage Form

### EXAMPLE 1

| Component | mg/cap |
|---|---|
| Valsartan | 80 |
| Lauroglycol FCC | 157.5 |
| EtOH | 52.5 |
| Tween 20 | 105 |
| Cremophor RH40 | 105 |

### EXAMPLE 2

| Component | mg/cap |
|---|---|
| Valsartan | 80 |
| Lauroglycol FCC | 157.5 |
| EtOH | 25 |
| PG | 37.5 |
| Tween 20 | 100 |
| Cremophor RH40 | 100 |

### EXAMPLE 3

| Ingredient | mg/dose |
|---|---|
| Valsartan | 80 |
| Lauroglycol FCC | 157.5 |
| Propylene Glycol | 62.5 |
| Tween 20 | 100 |
| Cremophor RH40 | 100 |

### EXAMPLE 4

| Ingredient | mg/dose |
|---|---|
| Valsartan | 80.00 |
| Lauroglycol FCC | 157.50 |
| Propylene Glycol | 61.70 |
| Tween 20 | 100.00 |
| Cremophor RH40 | 100.00 |
| Methyl Parabens | 0.72 |
| Propyl Parabens | 0.08 |

### EXAMPLE 5

| Ingredient | mg/unit |
|---|---|
| Valsartan | 80 |
| Lauroglycol FCC | 160 |
| Lauroglycol 90 | 50 |
| Tween 20 | 100 |
| Cremophor RH40 | 100 |

### EXAMPLE 6

| Ingredient | mg/unit |
|---|---|
| Valsartan | 80 |
| Spearmint oil | 150 |
| EtOH | 25 |
| PEG400 | 25 |
| Cremophor RH40 | 200 |

### EXAMPLE 7

| Ingredient | mg/unit |
|---|---|
| Valsartan | 80 |
| VitE TPGS | 150 |
| EtOH | 25 |
| PEG400 | 25 |
| Cremophor RH40 | 200 |

### EXAMPLE 8

| Ingredient | mg/unit |
|---|---|
| Valsartan | 80 |
| Maisine 35-1 | 150 |
| EtOH | 25 |
| PEG400 | 25 |
| Cremophor RH40 | 200 |

### EXAMPLE 9

| Ingredient | mg/unit |
|---|---|
| Valsartan | 80 |
| Maisine 35-1 | 150 |
| EtOH | 25 |
| PEG400 | 25 |
| Cremophor RH40 | 200 |

### EXAMPLE 10

| Ingredient | mg/unit |
|---|---|
| Valsartan | 80 |
| Labfill M2125 | 150 |
| EtOH | 80 |
| Cremophor RH40 | 200 |

### EXAMPLE 11

| Ingredient | mg/unit |
|---|---|
| Valsartan | 80 |
| Capmul PG8 | 150 |
| EtOH | 80 |
| Cremophor RH40 | 200 |

### EXAMPLE 12

| Ingredient | mg/unit |
|---|---|
| Valsartan | 80 |
| Capmul MCM | 150 |
| EtOH | 80 |
| Cremophor RH40 | 200 |

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A pharmaceutical composition comprising:
(a) valsartan; and
(b) a surfactant, a lipophilic component and a hydrophilic component.

2. The pharmaceutical composition of claim 1, wherein said pharmaceutical composition is liquid, solid or semisolid and forms a microemulsion when contacted with an aqueous medium.

3. The pharmaceutical composition of claim 1, wherein said lipophilic component is a liquid lipophilic component.

4. The pharmaceutical composition of claim 2, wherein said lipophilic component is an oil.

5. The pharmaceutical composition of claim 1, wherein said hydrophilic component is a solid polymer at room temperature.

6. The pharmaceutical composition of claim 1, wherein said hydrophilic component comprises a polyethylene glycol (PEG).

7. The pharmaceutical composition of claim 6, wherein said PEG is selected from the group consisting of PEG 1450, PEG 3350, PEG 4000, PEG 8000, derivatives and mixtures thereof.

8. The pharmaceutical composition of claim 1, wherein said pharmaceutical composition is an oral dosage form.

9. The pharmaceutical composition of claim 8, in the form of a capsule.

10. The pharmaceutical composition of claim 1, wherein said oil is an essential oil.

11. The pharmaceutical composition of claim 1, wherein said microemulsion comprises particles having a mean particle size less than 300 nm.

12. The pharmaceutical composition of claim 1, wherein said aqueous medium is a gastric juice.

13. The pharmaceutical composition of claim 1, wherein said surfactant and said lipophilic component are embedded in a polymeric matrix of the hydrophilic component.

14. The pharmaceutical composition of claim 1, further comprising a cosurfactant, filler, acidifier or a mixture thereof.

15. The pharmaceutical composition of claim 1, wherein said lipophilic component is a monoglyceride, diglyceride, triglyceride or a mixture thereof.

16. The pharmaceutical composition of claim 5, wherein said polymer comprises a polyethylene oxide.

17. A method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering a therapeutically effective amount of a pharmaceutical composition of claim 1 to a subject in need of such treatment.

18. A process for preparing a microemulsion containing a poorly soluble drug comprising the steps of:
(a) bringing valsartan and a liquefied carrier comprising a surfactant, a lipophilic component and a hydrophilic component into intimate admixture to form a microemulsion preconcentrate, said microemulsion preconcentrate is solid or semisolid at room temperature; and subsequently
(b) contacting said pharmaceutical composition with an aqueous medium to form a microemulsion.

19. The process of claim 18, wherein said lipophilic component is a liquid lipophilic component.

20. The process of claim 18, wherein said lipophilic component is an oil.

21. The process of claim 18, wherein said hydrophilic component comprises a PEG.

22. The process of claim 21, wherein said PEG is selected from the group consisting of PEG 1450, PEG 3350, PEG 4000, PEG 8000, derivatives thereof and mixtures thereof.

23. The process of claim 18, wherein said microemulsion comprises particles having a mean particle size less than 300 nm.

24. The process of claim 18, wherein said aqueous medium is a gastric juice.

25. The process of claim 18, wherein said surfactant and said lipophilic component are embedded in a polymeric matrix of said hydrophilic component.

26. The process of claim 18, wherein said carrier further comprises a cosurfactant.

27. The process of claim 18, wherein said lipophilic component is a monoglyceride, diglyceride, triglyceride or a mixture thereof.

28. The process of claim 18, wherein said hydrophilic component comprises polyethylene oxide.

29. A drug delivery system for a poorly soluble drug comprising a surfactant, a lipophilic component and a hydrophilic component, wherein said hydrophilic component consists essentially of a solid PEG.

30. The drug delivery system of claim 29, wherein said lipophilic component is an oil.
